(19) United States
(12) Patent Application Publication
(10) Pub. No.: US 2022/0162258 A1
(43) Pub. Date: May 26, 2022

(54) Method for Obtaining Gamma-Oryzanol

(71) Applicant: CPC Corporation, Taiwan, Taiwan (CN)

(72) Inventors: JUI-HUI CHEN, KAOHSIUNG (TW); YU-SIN JHAN, KAOHSIUNG (TW); AI-LING KAO, KAOHSIUNG (TW); CHIN-CHUNG CHEN, KAOHSIUNG (TW); CHANG-TING TSAI, KAOHSIUNG (TW); ZHENG-CHIA TSAI, KAOHSIUNG (TW)

(21) Appl. No.: 17/429,510

(22) PCT Filed: Jul. 29, 2020
(86) PCT No.: PCT/CN2020/105347

§ 371 Date: Aug. 9, 2021

(51) Int. Cl. C07J 53/00

(52) U.S. Cl. CPC C07J 53/004

(57) ABSTRACT

A method for obtaining γ-oryzanol comprises: (a) extracting an extract from a raw material containing γ-oryzanol by supercritical fluid extraction; (b) separating a pellet from the extract through solid-liquid separation; and (c) obtaining purified γ-oryzanol from the pellet.

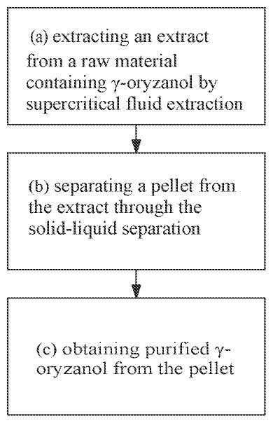

### BACKGROUND OF THE INVENTION

### 1. Field of the Invention

The present invention relates to a method for obtaining γ-oryzanol, and in particular, to a method for obtaining purified γ-oryzanol by supercritical fluid extraction.

### 2. Description of the Prior Art

γ-oryzanol is a compound formed by esterification of phytosterols, triterpenoids with ferulic acid. It is known that γ-oryzanol has antioxidant property to quench the free radicals generation, and can reduce serum lipid levels and alleviate menopausal symptoms.

γ-oryzanol has been widely used in food and cosmeceutical products. For example, γ-oryzanol can be added to foods to reduce the formation of cholesterol oxidation products (COPs), or γ-oryzanol can be added to creams to improve cells proliferation and increase skin elasticity and moisture.

γ-oryzanol is mainly extracted from vegetable oils. Crude rice bran oil contains the highest amounts of γ-oryzanol among the vegetable oils. Crude rice bran oil contains 1.4%-2.9% of γ-oryzanol, therefore, is served as a common raw material for γ-oryzanol preparation. In addition, other vegetable oils also contain γ-oryzanol, such as corn oil, soybean oil, which can also be used to prepare γ-oryzanol.

Generally, soapstock purification method is used to extract γ-oryzanol from crude rice bran oil. More specifically, the process of refining crude rice bran oil into refined oil includes degumming, deacidification, dewaxing, decolorization, and deodorization. In the deacidification step, the acidity of degummed crude oil is neutralized by alkali. During the deacidification process, soapstock will be obtained, and can be used for γ-oryzanol preparation. The process of the soapstock purification method includes: (a) homogenization: the soapstock containing 60-70% of water was stirred evenly under the conditions of 70-90° C. and pH10-11; (b) alkalization: adding 0.2-1.2% sodium hydroxide to the soapstock, and continuing to stir evenly; (c) saponification: stirring the soapstock at 70-90° C. for about 30˜45 minutes to obtain saponified soapstock; (d) dehydration: dehydrating the saponified soapstock; (e) dissolution: the dehydrated saponified soapstock was dissolved in a solvent; and (f) the solvent was removed to obtain the γ-oryzanol extract.

However, as described above, the process of preparing γ-oryzanol by the soapstock purification method requires many steps such as homogenization, alkalization, saponification, dehydration, and dissolution, so that the existing process of γ-oryzanol preparation is very complicated.

### SUMMARY OF THE INVENTION

To address the deficiencies of conventional process, an embodiment of the invention provides a method for obtaining γ-oryzanol, comprises: (a) extracting an extract from a raw material containing γ-oryzanol by supercritical fluid extraction; (b) separating a pellet from the extract through the solid-liquid separation; and (c) obtaining purified γ-oryzanol from the pellet.

In some embodiments, in step (a), the raw material containing γ-oryzanol is one or more kinds selected from a group consisting of rice, wheat, corn, linseed, rapeseed.

In some embodiments, when the raw material containing γ-oryzanol is selected from rice, the rice is one or more kinds selected from a group consisting of paddy rice, brown rice with the hull removed, rice bran or white rice with the bran layer removed, and the raw materials can be poured into the supercritical extraction tank individually or in mixture.

In some embodiments, in step (a), the extraction temperature of the supercritical fluid is between 30° C. and 90° C., and the extraction pressure is between 75 bar and 1000 bar.

In some embodiments, the extract is kept at a temperature in the range of 60 to 80° C. before solid-liquid separation.

In some embodiments, in step (b), a residue is filtered from the extract and dissolved in a non-polar solvent to become a slurry solution. The slurry solution is then separated by filtration or centrifugation, so that the pellet is obtained.

In some embodiments, in step (c), the pellet is dissolved in an organic solvent to become a γ-oryzanol-rich solution. Next, the γ-oryzanol-rich solution is filtered to become a ready-to-purify filtrate, so that when the organic solvent in the ready-to-purify filtrate is removed, the purified γ-oryzanol is obtained.

In some embodiments, the γ-oryzanol-rich solution is warmed to keep at a temperature in the range of 35-40° C. before filtration.

Through the method as described above, high-purity γ-oryzanol can be obtained at room temperature, and the whole process is simple, thereby solving the problem of the complicated process of the existing method for obtaining γ-oryzanol.

### BRIEF DESCRIPTION OF THE DRAWINGS

The FIGURE is a flow chart of a method for obtaining γ-oryzanol in accordance with an embodiment of this invention.

### DETAILED DESCRIPTION OF THE PREFERRED EMBODIMENTS

A preferred embodiment of the invention will be introduced with reference to appended figures as follows to demonstrate that the invention may be implemented. The configuration of each component in the specific embodiments discussed are merely for illustrative purpose, and do not limit the scope of the invention.

Referring to the FIGURE, a method for obtaining γ-oryzanol, comprises: (a) extracting an extract from a raw material containing γ-oryzanol by supercritical fluid extraction; (b) separating a pellet from the extract through the solid-liquid separation; and (c) obtaining purified γ-oryzanol from the pellet. Each of steps will be elaborated below.

Extracting an extract from a raw material containing γ-oryzanol by supercritical fluid extraction:

The brown rice was used as raw material, and about 12 tons of brown rice was filled in three series-connected ton-level extraction tanks (each extraction tank has a volume of 6 m³). The carbon dioxide was served as a supercritical fluid to extract oil from the brown rice. The extraction temperature was set at about 50° C., the extraction pressure was set at about 270 bar, and the extraction duration was 6-7 hours. The extract extracted from brown rice was dark brown oil with beige turbidity. After 8 batches of extraction, approximately 200 L of crude brown rice oil can be collected.

In the extraction step, the raw material containing γ-oryzanol could use paddy rice, brown rice, white rice, rice bran or a mixture thereof, but wheat, corn, or plant raw materials with high oil content such as linseed and rapeseed may also be used.

In the extraction step, the gasified carbon dioxide could be re-pressurized into liquid carbon dioxide as a supercritical fluid, and was reused and stored in the circulating pipeline.

In the extraction step, water, ethanol or other currently known suitable supercritical fluid materials could be used as co-solvents for carbon dioxide. Suitable fluid types other than carbon dioxide may also be selected. Besides, based on the process requirements or the properties of the supercritical fluid, the extraction temperature could be set at 30-90° C., and the extraction pressure was set at about 75-1000 bar. However, the temperature and pressure may be set in other numerical ranges depending on the process requirements or the properties of the supercritical fluid, and should not be construed to limit the scope of the present invention.

To improve the fluidity of the extract for a better subsequent separation, the temperature of the extract was maintained at 80° C. through an oven to keep the extract in a fluid dark brown grease state. However, the extract may also be maintained in other temperature ranges to be in a fluid state based on different requirements, for example, between 60° C. and 80° C.

Separating a pellet from the extract through the solid-liquid separation:

The extract was subjected to solid-liquid separation. In the embodiment, the extract was filtered with filter paper to separate the dark brown filtrate from the beige filter residue, and the beige filter residue contained γ-oryzanol. Next step was to clean the filter residue. The filter residue was dissolved in the non-polar solvent n-hexane to become a slurry solution, and then the slurry solution was subjected to centrifugal separation, so that a pellet and a supernatant were obtained. If the supernatant appeared light yellow, new n-hexane was added to clean the pellet again. Repeatedly cleaned the filter residue with n-hexane until the supernatant obtained from the centrifuged slurry solution appeared colorless. Colorless and clear means that the filter residue was fully cleaned.

In the embodiment, the pellet was separated from the slurry solution by centrifugation, but the pellet may also be separated by filtration or other known solid-liquid separation methods.

The n-hexane supernatant was collected and recovered by distillation under reduced pressure and can be reused again. The pellet cleaned by n-hexane could be placed in a chemical extraction cabinet for drying, so that the n-hexane in the pellet could be completely volatilized, or the n-hexane in the pellet may also be drawn out by vacuum decompression.

In the embodiment, n-hexane was selected as the non-polar solvent, but non-polar solvents with similar properties such as n-heptane, linear or cyclic hydrocarbon solvents may also be used.

In the embodiment, to fully remove the impurities in the pellet for the later purification of γ-oryzanol, the steps of filtering the extract and cleaning the filter residue were carried out in a sequential manner. However, in other embodiments, it is also possible to perform only the step of filtering the extract, and the filter residue was directly served as the pellet for subsequent purification of γ-oryzanol.

Obtaining Purified γ-oryzanol from the Pellet:

The pellet contains insoluble substances like impurities, debris. To obtain high-purity γ-oryzanol, in this step, acetone was used to dissolve out γ-oryzanol from the pellet. That is, γ-oryzanol in the pellet was dissolved in acetone to become a γ-oryzanol-rich solution. The dissolution of γ-oryzanol was monitored by thin layer chromatography (TLC) until γ-oryzanol in the pellet was no longer dissolved. Next, the γ-oryzanol-rich solution was warmed and maintained at 35-40° C. to avoid the premature precipitation of γ-oryzanol caused by the temperature drop during filtration, thereby reducing the amount of recovered γ-oryzanol. However, in other embodiments, this warming step may also be omitted. After the γ-oryzanol-rich solution was warmed, and followed by the filtration, a clear acetone filtrate was obtained and served as a ready-to-purify filtrate. However, in other embodiments, the filtration of the γ-oryzanol-rich solution may be omitted, such as the γ-oryzanol-rich solution is clear enough and contains no debris. Finally, the ready-to-purify filtrate was distilled under reduced pressure to recover the acetone, and γ-oryzanol was precipitated while the acetone was removed. Alternatively, the ready-to-purify filtrate may be placed in a low temperature environment to precipitate γ-oryzanol, and then γ-oryzanol could be obtained by solid-liquid separation at low temperature. The precipitated γ-oryzanol was presented in powder form. To remove the residual solvent, the precipitated γ-oryzanol could be placed in an oven and baked at 50° C. overnight.

In the embodiment, acetone was used to dissolve out γ-oryzanol from the pellet, but ethyl acetate, isopropanol, ethanol, methanol, mixtures thereof, or organic solvents with similar properties may also be used.

Qualitative and Quantitative Test of γ-oryzanol:

To determine the purity of the obtained γ-oryzanol, the final sample powder prepared by the embodiments was determined by following several qualitative and quantitative techniques.

Qualitative Test by Potassium Hydroxide-Ethanol:

0.01 g of the sample powder was dissolved in 10 mL of potassium hydroxide-ethanol solution (10%, w/v). If the solution appears yellow, there is γ-oryzanol in the solution. The test result was that the solution appeared yellow.

Qualitative Test by Ferric Chloride:

0.01 g of sample powder was dissolved in 2 mL of acetone solution, followed by addition of 0.1 mL of ferric chloride ethanol (2%, w/v). If the solution appears yellow-green or green, there is γ-oryzanol in the solution. The test result was that the solution appeared yellow-green.

Residual Solvent:

In the embodiment, Headspace GC-MS was used to determine the residual solvent in the sample powder. The test result was that there was no solvent left in the sample powder.

Moisture Content Test:

In the embodiment, the moisture content of the sample powder was measured by weight loss upon drying, and the test result was that the sample power had about 0.32% moisture content. Therefore, there were relatively small amounts of moisture content in the sample powder.

Inorganic Impurities Testing:

In the embodiment, the residue on ignition test was used to determine the amount of inorganic impurities in the sample powder. The mass of inorganic impurities in the sample powder to the total mass is 2.53%. Therefore, there were very few inorganic impurities in the sample powder.

Heavy Metal Testing:

In the embodiment, according to the “Methods for Identification and Determination of Lead, Cadmium, and Arsenic in Cosmetics” and “Methods for Identification and Determination of Mercury in Cosmetics” published by the Food and Drug Administration of the Ministry of Health and Welfare of Taiwan, inductively coupled plasma mass spectrometry (ICP-MS) was used to determine whether the sample powder contained heavy metals. The test result was that no heavy metal substances such as lead (Pb), arsenic (As), cadmium (Cd), mercury (Hg) were detected in the sample powder.

Microbiology Testing:

In the embodiment, the rapid plates for microbial tests (i.e., 3M Petrifilm AC, RYM, EC, STX, and CompactDry PA) were used to determine the total number of bacteria and fungi in the sample powder, and to determine whether the sample powder contained Escherichia coli, Staphylococcus aureus and Pseudomonas aeruginosa. After testing, no microorganisms were observed in the sample powder.

γ-oryzanol Purity Test:

0.025 g of sample powder was placed in a 5 mL volumetric flask, followed by addition of 0.5 mL of acetone to dissolve the sample powder. Then, n-heptane was added to the 5 mL volumetric flask to make a total volume of 5 mL, which was served as a pretreatment solution. Take another 25 ml volumetric flask, and pour n-heptane to almost 90% full. Then, 50 μL of pretreatment solution was added to the 25 ml volumetric flask, and followed by addition of n-heptane to make a total volume of 25 mL, which was served as a test solution. The absorbance of the test solution was measured by a spectrophotometer at 315 nm, and γ-oryzanol was calculated by the following formula: γ-oryzanol (%)=(A×2500)/(W*E) (W is weight of the sample g; A is absorbance of the sample at 315 nm; E is absorption coefficient 1% (1 cm light path)=359). Finally, the sample powder had >95% of γ-oryzanol content, which proved that the purity of γ-oryzanol obtained by the embodiments of present invention could reach more than 95%.

In addition, when the test solution was used to obtain the spectral absorption profile, the result showed maximum absorbances in the range of 229-233 nm, 289-293 nm, and 313-317 nm. The above absorption spectrum also proved that the test solution contained γ-oryzanol.

The present invention can produce high-purity γ-oryzanol at room temperature, and the purity of γ-oryzanol can reach more than 95%. Besides, the whole process is simple, and the homogenization, alkalization, saponification, dehydration, dissolution, and dissolution required by soapstock purification method can be omitted, thereby solving the problem of complicated manufacturing process of the existing method for obtaining γ-oryzanol. In addition, compared with the existing soapstock purification method, the present invention generates less waste, and the solvent used in the preparation process of the present invention can be distilled, recovered, and reused.

Although various embodiments have been described for illustrative purposes, it is obvious to those skilled in the art that various changes and improvements can be made without departing from the spirit and scope of the invention as defined in the appended claims

What is claimed is:

1. A method for obtaining γ-oryzanol, comprising:
(a) extracting an extract from a raw material containing γ-oryzanol by supercritical fluid extraction;
(b) separating a pellet from the extract through the solid-liquid separation; and
(c) obtaining purified γ-oryzanol from the pellet.

2. The method for obtaining γ-oryzanol as claimed in claim 1, wherein in step (a), the raw material containing γ-oryzanol is one or more kinds selected from a group consisting of rice, wheat, corn, linseed, rapeseed.

3. The method for obtaining γ-oryzanol as claimed in claim 2, wherein when the raw material containing γ-oryzanol is selected from rice, the rice is one or more kinds selected from a group consisting of paddy rice, brown rice with the hull removed, rice bran or white rice with the bran layer removed.

4. The method for obtaining γ-oryzanol as claimed in claim 1, wherein in step (a), the extraction temperature of the supercritical fluid is between 30° C. and 90° C., and the extraction pressure is between 75 bar and 1000 bar.

5. The method for obtaining γ-oryzanol as claimed in claim 1, wherein in step (b), the extract is kept at a temperature in the range of 60 to 80° C. before solid-liquid separation.

6. The method for obtaining γ-oryzanol as claimed in claim 1, wherein in step (b), a residue is filtered from the extract and dissolved in a non-polar solvent to become a slurry solution, and then the pellet is separated from the slurry solution.

7. The method for obtaining γ-oryzanol as claimed in claim 1, wherein in step (c), the pellet is dissolved in an organic solvent to become a γ-oryzanol-rich solution, the γ-oryzanol-rich solution is filtered to become a ready-to-purify filtrate, and when the organic solvent in the ready-to-purify filtrate is removed, the purified γ-oryzanol is obtained.

8. The method for obtaining γ-oryzanol as claimed in claim 7, wherein the γ-oryzanol-rich solution is warmed to keep at a temperature in the range of 35-40° C. before filtration.
